(19) United States
(12) Patent Application Publication
(10) Pub. No.: US 2025/0082659 A1
(43) Pub. Date: Mar. 13, 2025

(54) MANNO-OLIGOSACCHARIDE COMPOSITIONS FOR PATHOGEN CONTROL

(71) Applicant: GREENSAGE PREBIOTICS INC., Oakville (CA)

(72) Inventor: Bradley Arthur SAVILLE, Oakville (CA)

(21) Appl. No.: 18/569,494

(22) PCT Filed: Aug. 15, 2023
(86) PCT No.: PCT/CA2023/051080

§ 371 Date: Dec. 12, 2023

Related U.S. Application Data
(60) Provisional application No. 63/397,993, filed on Aug. 15, 2022.

(51) Int. Cl. A61K 31/702; A61K 31/43; A61K 31/7004; A61K 31/716; A61K 45/06; A61P 31/04

(52) U.S. Cl. CPC A61K 31/702; A61K 31/43; A61K 31/7004; A61K 31/716; A61K 45/06; A61P 31/04

(57) ABSTRACT

The present application includes a composition comprising manno-oligosaccharide (MOS) carbohydrates for use in inhibiting growth of pathogenic bacteria and/or promoting growth of beneficial bacteria in a subject, wherein at least 70% wt of the MOS carbohydrates are mannose sub-units. The MOS is derived from mannan material provided from plant sources. The composition has a low average DP which provides improved solubility of the composition. The present application also includes a method of preparing the composition.

### RELATED APPLICATION

This application claims the benefit of priority from U.S. Provisional Patent Application No. 63/397,993 filed on Aug. 15, 2022, the contents of which are incorporated herein by reference in their entirety.

### FIELD

This application pertains to the use of manno-oligosaccharide compositions for prevention and treatment of infections. In particular, the present application pertains to highly pure manno-oligosaccharide compositions for prevention and treatment of infections caused by, for example, pathogenic bacteria and method of preparing the same.

### BACKGROUND

Infections in animals and humans are leading cause of illness and death. In production animals such as poultry, livestock, and fish, infections may reduce the growth rate, reduce the efficiency of feed utilization, and increase mortality. In humans and companion animals, infections of, for example, the digestive and urinary tract, affect health, quality of life, and in severe cases, may cause death.

Bacterial infections in production animals have historically been treated by antibiotics, and in many cases, antibiotics have been delivered prophylactically as growth promoters. While this approach is effective, it has led to concerns about overuse of antibiotics, antimicrobial resistance, and antibiotic residues in meat products. Overuse of antibiotics has been highlighted by the World Health Organization as a major challenge to be addressed, with potentially severe consequences for infection control.

Recurrent bacterial infections in humans, for example, in the digestive and urinary tracts, have also been treated with long term antibiotics, resulting in adverse effects on the beneficial bacteria, increased risk of antibiotic-associated diarrhea and increased risk of antibiotic-resistant infections such as Clostridioides difficile (C. difficile).

There is a clear need to develop alternatives to antibiotics for prevention and treatment of infections in animals and humans, while reserving the use of antibiotics for treatment of more severe cases. Various options have been under consideration, including (i) compounds such as phages and quorum sensing that may target microbes, (ii) compounds such as probiotics, prebiotics, polyphenols and fatty acids that support the immune system either directly or indirectly via metabolites, and help resist the adverse impacts of pathogens, and (iii) compounds such as probiotics and prebiotics that may limit the growth of pathogenic bacteria by increasing the amount of beneficial bacteria and crowding out pathogenic bacteria (Principi, N., et al., Advantages and Limitations of Bacteriophages for the Treatment of Bacterial Infections, Front. Pharmacol., 8 May 2019; Jiang, Q., et al., Quorum Sensing: A Prospective Therapeutic Target for Bacterial Diseases, Biomed Res. Apr. 4, 2019).

Common prebiotics include fructo-oligosaccharides (FOS), inulin, galacto-oligosaccharides (GOS), and xylo-oligosaccharides (XOS). These prebiotics contain sub-units comprised of fructose, fructose, galactose, and xylose, respectively. The nature of the bonds between these sub-units depends upon their source and how they are produced. The bond structure, along with the degree of polymerization, may substantially affect their interactions with bacteria, by binding, transport, and/or metabolism.

Data from Makelainen et al. (Makelainen H. et al., Xylo-oligosaccharides and lactitol promote the growth of Bifidobacterium lactis and Lactobacillus species in pure cultures, Beneficial Microbes, 2010; 1:139-148) indicate that common prebiotics promote the growth of common pathogens, including Enterohemorrhagic Escherichia coli (EHEC), Salmonella typhimurium, Clostridium perfringens, Staphylococcus epidermis (FIG. 1). Makelainen shows that pathogens grow significantly in the presence of GOS and FOS, while there is less growth with short chain xylo-oligosaccharides (scXOS) and XOS with a degree of polymerization (DP) of 2-16. Pathogen growth clearly varies by species, and there are significant differences between carbon sources, reflecting differences in the primary carbohydrate subunit (fructose, glucose, galactose, xylose), the types of bonds between sub-units, and degree of polymerization. Nonetheless, among these pathogens studied, there is little indication of pathogen growth inhibition due to these prebiotics.

Oligosaccharides and polysaccharides derived from mannan have also been proposed as prebiotics, although unlike the prebiotics listed above, there has been limited use in humans, for the reasons described below. Long chain mannan-oligosaccharides/polysaccharides are derived from the cell walls of yeast, where these oligosaccharides/polysaccharides are cross-linked to β-glucan and protein. These long chain mannan-oligosaccharides/polysaccharide fractions from yeast have α, 1-4 linkages, and typically have a high degree of polymerization (DP), from fifty to hundreds. Although often described in the literature as mannan-oligosaccharides or manno-oligosaccharides (MOS), they are rigorously defined as polysaccharides due to their high degree of polymerization. The high degree of polymerization renders these mannan fractions sparingly soluble in water. AgriMOS, for example, is stated to have a solubility of 8%, per their product monograph (The Blocking Effect on Undesirable Bacteria, Product Monograph from Lallemand Animal Nutrition, lallemandanimalnutrition.com). Poly/oligosaccharides from mannan in yeast (α-MOS) are generally present in low purity, from 10-30 weight percent α-mannan, along with 10-30 wt % β-glucan, and up to 30% protein (The Blocking Effect on Undesirable Bacteria, Product Monograph from Lallemand Animal Nutrition, lallemandanimalnutrition.com). The low purity and complex bond structure of these yeast-derived products makes it difficult to determine the mechanism of action, since both α-MOS and β-glucan are claimed to have immune-enhancing benefits (The Blocking Effect on Undesirable Bacteria, Product t Monograph from Lallemand Animal Nutrition, lallemandanimalnutrition.com). Although these yeast-derived “MOS” products have been used in poultry and livestock, the results have been variable, possibly due to variability in the composition, and the complex cross-linking between α-mannan/α-MOS, β-glucan, protein, and other constituents such as ash and other fibers.

These low purity α-mannan/α-MOS products have been used as a natural growth promoter in poultry and livestock, supporting the immune system and reducing susceptibility to infections caused by, e.g., Salmonella and E. coli. These benefits may be indirect, arising from the production of short-chain fatty acids and other metabolites by beneficial bacteria stimulated by the α-MOS and β-glucan components. There may also be some direct impacts on pathogens by inhibition of binding by Type I fimbriae that facilitate adhesion to mannose-containing receptors (lectins) that line the digestive tract, urinary tract, and other tissues in animals and humans. The low purity products containing β-glucan, α-mannan/α-MOS and protein have been suggested to facilitate agglutination of microbes. However, the high degree of polymerization of α-mannan/α-MOS, the cross-linking of α-mannan/α-MOS to β-glucan and protein, and the low (or no) solubility of the α-mannan/α-MOS may affect the efficacy of these yeast-derived products for pathogen binding/agglutination, and lead to variable immune responses and health benefits. Furthermore, it has not been established whether the proposed benefits versus certain pathogens are due to the α-mannan, β-glucan, or other components in these mixtures. Variability in the yeast source, composition and processing method may affect the α-mannan, β-glucan, and protein content, and the degree of polymerization (DP) of the oligo-/polysaccharides and contribute to variability in results and lack of efficacy of these products for certain applications. Existing data with “MOS” in animals are based on these low purity α-mannan/α-MOS/β-glucan/protein products.

Several studies such as Ariandi et al. (Ariandi, Y., et al., Enzymatic Hydrolysis of Copra Meal by Mannanase from Streptomyces sp. BF3.1 for The Production of Mannooligosaccharides, HATAYI Journal of Biosciences, Vol 22 (2), PP 79-86, 2015), Cuong et al. (Cuong, D. B. et al., Bioconversion Of Copra Meal Into Prebiotic Mannooligosaccharides Using Endo-B-1,4-Mannanase Producing By Aspergillus Niger Bk 01, Science and Technology Journal, vol 48 (3), p 43-49 2010) and Rungrassamee et al. (Rungrassamee, W., et al., Mannooligosaccharides from copra meal improves survival of the Pacific white shrimp (Litopenaeus vannamei) produced only low purity β-MOS, with other compounds present. The influence of these other compounds on the applications of the β-MOS has not yet been determined.

Therefore, there is an unmet need for compositions which effectively inhibit the growth of pathogenic bacteria in animals and humans, thereby reducing the use of antibiotics.

### SUMMARY

The present application discloses a high purity, β-MOS composition for use in inhibiting the growth of pathogenic bacteria. The composition of the present application improves survival and growth of production animals and helps treating infections in humans, thereby reducing the use of antibiotics.

Accordingly, the present application includes a composition comprising manno-oligosaccharide (MOS) carbohydrates for use in inhibiting growth of pathogenic bacteria and/or promoting growth of beneficial bacteria in a subject, wherein at least 70% of the MOS carbohydrates are mannose sub-units.

The present application also includes a combination of manno-oligosaccharide (MOS) carbohydrates composition and an antibiotic, wherein the efficacy of the antibiotic is increased by at least 30%.

The present application also includes a method of producing MOS carbohydrates composition comprising subjecting mannan material to hydrolysis to obtain a crude extract, and purifying the crude extract to obtain a purified extract, wherein at least 70% wt of the MOS carbohydrates are mannose sub-units.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1: shows the aggregate growth of select pathogens when grown on glucose (control) and select prebiotics. Pathogen growth is represented by the optical density (OD), and the cumulative pathogen growth is represented by the area under the curve (AUC), equivalent to the OD x time. A high area under the curve indicates high level of growth; and a negative area under the curve suggests potential inhibition.

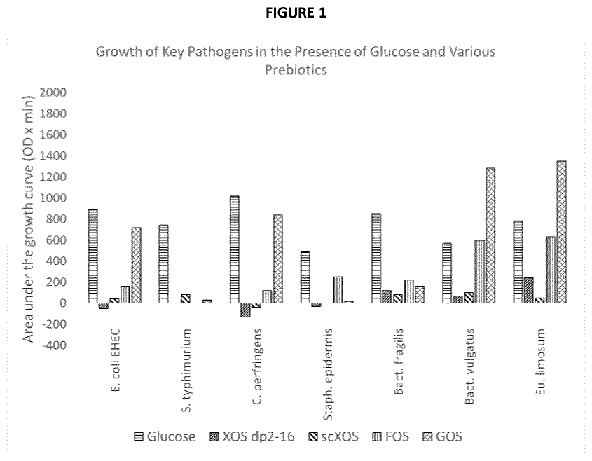

FIG. 2: shows the high-performance liquid chromatogram (HPLC) identifying and quantifying the carbohydrate components of the exemplary MOS purified extract solution from Copra Meal.

FIG. 3: shows inhibition of Salmonella enteritidis in the presence of exemplary β-manno-oligosaccharides (β-MOS) from Copra Meal optical density (OD) measured every 2 h over 24 h at 630 nm versus equivalent doses in feed of 0.05 wt % to 0.25 wt %.

FIG. 4: shows delta optical density (OD₆₀₀) values versus compound concentrations (mg/ml) for 6 dilutions of exemplary compound solutions copra-MOS (CMOS), yeast-MOS (YMOS), and YMOS NaOH measured at a wavelength of 600 nm, for minimum inhibitory concentration (MIC) determination of Vibrio parahaemolyticus. Statistically significant (p<0.05, growth inhibition>20%) values are shown with an asterisk (*). The negative control is 0 mg/ml of compound in either sterile distilled water (CMOS, YMOS) or 0.4% 1M NaOH solution (YMOS NaOH). The grey dashed line separates each dilution group. The labels across the top of each section describe the compound solutions. In every section, the first result refers to CMOS compound solution (C), the second result refers to YMOS compound solution (Y), and the third result refers to YMOS NaOH (Y-NaOH) compound solution.

FIG. 5: shows inhibition of Piscirickettsia salmonis in the presence of exemplary β-manno-oligosaccharides (C-MOS) from Copra Meal at concentration of 5.5, 11.1, 16.7 or 25 mg/ml C-MOS, measured after 11 and 16 days of incubation.

FIG. 6: shows inhibition of P. salmonis in the presence of yeast MOS (Y-MOS), at concentration of 5.5, 11.1 or 16.7 mg/ml Y-MOS, measured after 11 and 16 days of incubation.

### DESCRIPTION OF VARIOUS EMBODIMENTS

### Definitions

Unless otherwise indicated, the definitions and embodiments described in this and other sections are intended to be applicable to all embodiments and aspects of the present application herein described for which they are suitable as would be understood by a person skilled in the art.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “composition(s) of the application” as used herein refers to a composition comprising manno-oligosaccharide (MOS) carbohydrates of the application.

The term “combination composition(s) of the application” as used herein refers to a composition comprising manno-oligosaccharide (MOS) carbohydrates of the application and antibiotics.

The term “method of the application” as used herein refers to a method of preparing the composition(s) of the application.

The term “monosaccharide” as used herein refers to a simple sugar that constitutes the building blocks of a more complex form of sugars such as oligosaccharides and polysaccharides.

The term “polysaccharide” as used herein refers to a carbohydrate formed by long chains composed of repeating monosaccharide units linked together by glycosidic bonds.

The term “oligosaccharides” as used herein refers to polymers of monosaccharides that have a degree of polymerization (DP) of 2 to 10.

The term “manno-oligosaccharides (MOSs)” as used herein refers to polysaccharides that include mannose monosaccharide residues. The mannose residues may be in the form of D-mannose, galactomannan, glucomannan, and mixtures thereof. The polysaccharides that include mannose may be entirely formed of mannose sub-units or may be a combination of mannose monosaccharide residues and other monosaccharides, such as for example, galactose, glucose and fructose. manno-oligosaccharides may include a plurality of oligosaccharides with different degrees of polymerization. The polysaccharides may be α-MOS or β-MOS.

The term “pathogenic bacteria” as used herein refers to a bacteria that can cause a disease in a subject. Examples of pathogenic bacteria include, but are not limited to, Vibrio, Tenacibaculum, Clostridia, Salmonella, Escherichia coli and Piscirickettsia salmonis or pathogenic bacteria containing Type I fimbriae.

The term “beneficial bacteria” as used herein refers to bacteria that are considered to provide health benefits, such as Lactobacillus spp., and Bifidobacteria spp.

The term “subject” as used herein refers to all members of animal kingdom. Thus, the uses of the present applications are applicable to both humans and animals.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e., not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. Treatment methods comprise administering to a subject a therapeutically effective amount of the composition or combination composition of the application and optionally consist of a single administration, or alternatively comprise a series of administrations. For example, in some embodiments, the compositions or combination compositions of the application may be administered at least once a week. In some embodiments, the compositions may be administered to the subject from about one time per three weeks, or about one time per week to about once daily for a given treatment. In another embodiment, the compositions are administered 2, 3, 4, 5 or 6 times daily. The length of the treatment period depends on a variety of factors, such as the severity of the disease, disorder or condition, the age of the subject, the concentration and/or the activity of the composition of the application, and/or a combination thereof. It will also be appreciated that the effective dosage of the composition used for the treatment may increase or decrease over the course of a particular treatment regime. Changes in dosage may result and become apparent by standard diagnostic assays known in the art. In some instances, chronic administration may be required. For example, the compositions are administered to the subject in an amount and for duration sufficient to treat the patient.

The term “prevention” or “prophylaxis”, or synonym thereto, as used herein refers to a reduction in the risk or probability of a patient becoming afflicted with a disease, disorder or condition mediated by pathogenic bacteria or treatable by inhibition of pathogenic bacteria, or manifesting a symptom associated with a disease, disorder or condition mediated by pathogenic bacteria.

The term “disease, disorder or condition mediated by pathogenic bacteria” as used herein refers to a disease, disorder or condition treatable by inhibition of pathogenic bacteria activity or promoting growth of beneficial bacteria, such as a bacterial infection.

The term “to inhibit growth of pathogenic bacteria” and variations thereof as used herein means any detectable inhibition of the growth of or killing of the pathogenic bacteria in the presence of a composition or combination composition of the application compared to otherwise the same conditions except in the absence of the composition of the application.

The term “to promote growth of beneficial bacteria” and variations thereof as used herein means any detectable promotion of the growth of the beneficial bacteria in the presence of a composition or combination composition of the application compared to otherwise the same conditions except in the absence of the composition of the application.

As used herein, the term “effective amount” means an amount of a composition or combination composition of the application that is effective to achieve the desired result. For example in the context of inhibiting growth of pathogenic bacteria while promoting growth of beneficial bacteria, an effective amount is an amount that, for example, increases said inhibition of pathogenic bacteria while promoting growth of said beneficial bacteria, compared to the same conditions except in the absence of the composition of the application.

The term “degree of polymerization (DP)” as used herein refers to the number of monosaccharides constituting an oligosaccharide or polysaccharide. Therefore, for example, the degree of polymerization of a manno-oligosaccharide in which mannose is composed of four monosaccharides is 4, and therefore, it is described as DP4.

The term “β-1,4 linkage” and “α, 1-4 linkages” as used herein refers to a bond of oxygen to the C₁ carbon of one carbohydrate ring structure and to the C₄ carbon of another carbohydrate ring structure, in the beta configuration. Beta configuration is distinct from an alpha configuration based upon the position of the bound hydroxyl group on the two carbohydrate rings. In the beta bond configuration, the hydroxyl group of C₁ is above the plane of the carbohydrate ring, while in an alpha bond configuration, the hydroxyl group of C₁ is below the plane of the carbohydrate ring.

The term “probiotics” as used herein refers to live microorganisms that, when administered in adequate amounts, confer a health benefit on the host (internationalprobiotics.org).

The term “prebiotics” as used herein refers to a substrate that is selectively utilized by host microorganisms conferring a health benefit (isappscience.org).

The term “polyphenols” as used herein refers to plant-derived organic compounds that contain one or more phenolic groups.

The term “phages” as used herein refers to a virus that infects and replicates within and destroys bacteria.

The term “clays and minerals” as used herein refers to a fine-grained soil material, usually derived from hydrolysis of feldspar to produce kaolinites and smectites.

The term “antibiotics” as used herein refers to a medicine that inhibits the growth of or destroys bacterial organisms.

The term “growth promoters” refers to substances that are added to feeds as supplement or injection to improve feed utilization and growth of animals.

The term “short chain fatty acids” as used herein refers to fatty acids with fewer than 6 carbon atoms. Examples include acetic acid, propionic acid, butyric acid and the like.

The term “mannan material” as used herein refers to raw material that contains mannan. Examples include but are not limited to residues from palm and coconut processing, copra meal, softwoods such as pine or spruce, residuals from coffee processing, and acai seeds and residues.

The term “enzyme” as used herein refers to a protein that acts as a biological catalyst for a reaction.

The term “TSB” as used herein refers to tris buffered saline.

The term “CFS” as used herein refers to cell free supernatant.

The term “OD” as used herein refers to optical density.

The term “CFU” as used herein refers to colony forming unit/ml.

The term “MA/MB media” as used herein refers to marine agar/marine broth.

The term “TSA2/TSB2 media” as used herein refers to tryptone soy agar/tryptone soy broth.

The term “MIC” as used herein refers to minimum inhibitory concentration.

The term “MLC” as used herein refers to minimum lethal concentration.

The term “FCR” as used herein refers to feed conversion ratio.

The term “wt” as used herein refers to weight.

### Compositions of the Application

The present application includes a composition comprising manno-oligosaccharide (MOS) carbohydrates for use in inhibiting growth of pathogenic bacteria and/or promoting growth of beneficial bacteria in a subject, wherein at least 70% wt of the MOS carbohydrates are mannose sub-units.

In some embodiments, the beneficial bacteria include, e.g., Lactobacillus spp. and Bifidobacteria spp. In some embodiments, the composition increases the growth of the beneficial bacteria by at least 10%, by at least 30%, at least 50% or at least 75% and values therebetween.

In some embodiments, the MOS is derived from mannan material. In some embodiments, the mannan material is provided from plant sources including but are not limited to palm kernel cake, coconut residue, softwoods such as pine or spruce, residuals from coffee processing, acai seeds and residues, copra meal and the like. As such, in some embodiments, the MOS is derived from mannan material provided from plant sources selected from palm kernel cake, coconut residue, softwoods such as pine or spruce, residuals from coffee processing, acai seeds and residues, and copra meal. In some embodiments, the mannan material is provided from copra meal.

In some embodiments, the degree of polymerization (DP) of the MOS is less than 20. In some embodiments, the DP of the MOS is less than 8, or from 1 to 8. In some embodiments, the DP is from 2 to 10. In some embodiments, the DP is from 2 to 6. In one embodiment, the DP is 2, 3, 4, 5, or 6. In one embodiment, the composition comprises manno-oligosaccharides having different degrees of polymerization. For example, a portion of the MOS may have a DP of 2, while another portion of the MOS has a DP of 4.

In some embodiments, the MOS having DP of 2 is present in the composition at a content of over 50 wt %. In some embodiments, the MOS having DP of 2 is present in the composition at a content of about 60 wt %, about 65 wt %, about 70 wt %, or about 80 wt %, and values therebetween. In some embodiments, the MOS derived from plant sources provides in a high content of MOS having DP of 2.

In some embodiments, at least 75% wt of the MOS carbohydrates are mannose sub-units. In some embodiments, at least 80% wt of the MOS carbohydrates are mannose sub-units. In some embodiments, at least 85% wt, or at least 90% wt, or at least 95% wt, of the MOS carbohydrates are mannose sub-units. In some embodiments, at least 85% wt of the MOS carbohydrates are mannose sub-units.

In some embodiments, the composition of the present application has a water solubility of above 90% at 15 wt % to 25 wt % aqueous solution of the composition at 25° C. In some embodiments, the composition has a water solubility of above 95% at 15 wt % to 25 wt % aqueous solution of the composition at 25° C. In some embodiments, the composition has a water solubility of about 95% to about 100% at 15 wt % to 25 wt % aqueous solution of the composition at 25° C. In some embodiments, the composition has a water solubility at least 95%, at least 96%, at least 97%, at least 98%, or at least 99% at 15 wt % to 25 wt % aqueous solution of the composition at 25° C. In some embodiments, the composition has a water solubility of about 100% at 15 wt % to 25 wt % aqueous solution of the composition at 25° C. Methods to detect and/or quantify water solubility are well known in the art. In some embodiments, the composition of the application wherein the MOS having DP of 2 is present in the composition at a content of over 50 wt % provides improved solubility of the composition.

In some embodiments, the mannose sub-units comprise predominantly β-1,4 linkages. In some embodiments, the mannose sub-units comprise α, 1-4 linkages. In some embodiments, the β-1,4 linkages of the mannose sub-units are well suited for the utilization by the beneficial bacteria. As such, in some embodiments, the amount of the beneficial bacteria will increase, and may crowd out pathogenic bacteria. The beneficial bacteria are any beneficial bacteria known in the art, such as for example probiotics which include microorganisms such as Lactobacillus, Bifidobacterium, Saccharomyces, Streptococcus, Enterococcus, Escherichia, and Bacillus and the like. In some embodiments, the beneficial bacteria include microorganisms that contain endo, b-1,4 mannanases.

In some embodiments, the composition further comprising at least one monosaccharide selected from the group consisting of glucose, galactose, xylose, arabinose, and combinations thereof.

In some embodiments, the content of glucose is less than 10% wt of the MOS. In some embodiments, the content of glucose is less than 8% wt of the MOS. In some embodiments, the content of glucose is between 3 and 7% wt of the MOS. In some embodiments, the content of glucose is less than 3% wt of the MOS.

In some embodiments, the content of galactose is less than 5% wt of the MOS. In some embodiments, the content of galactose is between 1 and 3% wt of the weight of the MOS. In some embodiments, the content of galactose is less than 1% wt of the MOS.

In some embodiments, the composition further comprising glucose and galactose. In some embodiments, the total glucose and galactose content is less than 10% wt of the weight of the MOS.

In some embodiments, the total monosaccharide content is less than 15% wt of the MOS. In some embodiments, the total monosaccharide content is less than 13% wt of the MOS. In some embodiments, the total monosaccharide content is less than 10% wt of the MOS.

In some embodiments, the composition of the present application is free from fructose.

In some embodiments, the composition further comprises β-glucan, wherein the content of the β-glucan is from about 0.5% wt to about 5% wt of the MOS. In some embodiments, the composition further comprises β-glucan wherein the content of the β-glucan is about 1%, about 2%, about 3%, or about 4% of the MOS, and values therebetween. In one embodiment, it has been found that high purity MOS, even with low levels of from about 0.5% wt to about 5% wt beta-glucan, as compared to yeast-based MOS which is typically >20 wt % (known to support immunity), is effective for pathogen inhibition, and effective on a broad spectrum of pathogens, including those that do not rely on mannose-containing lectins, nor rely on promotion of agglutination.

In some embodiments, the composition further comprising an agent selected from the group consisting of probiotics, prebiotics, polyphenols, phages, clays and minerals such as bentonite and montmorillonite, antibiotics and short chain fatty acids.

The probiotics include microorganisms such as Lactobacillus, Bifidobacterium, Saccharomyces, Streptococcus, Enterococcus, Escherichia, and Bacillus and the like.

The prebiotics include fructo-oligosaccharides (FOS), inulin, galacto-oligosaccharides (GOS), and xylo-oligosaccharides (XOS) and the like.

Polyphenols include but are not limited to flavonoids, lignans, stilbenes, phenolic acids and the like.

A person skilled in the art would understand that phages are specific to a particular pathogen. The selection of particular phages is within the purview of the person skilled in the art.

The antibiotics include any antibiotics known in the art. Examples of antibiotics include but are not limited to penicillin, gentamycin, clindamycin, ceftazidime and the like.

The short chain fatty acids include fatty acids with fewer than 6 carbon acids. Examples of short chain fatty acids include but are not limited to, acetic acid, propionic acid, butyric acid and the like.

In some embodiments, the composition is in a form of an aqueous solution or powder.

In some embodiments, the aqueous solution or powder is provided by hydrolysis and purification of a mannan material.

In some embodiments, the composition is formulated for oral administration.

In some embodiments, the composition is formulated in a form of a supplement, food, beverage or feed additive.

In some embodiments, the composition is formulated in a form of a capsule, tablet, sachet, or liquid.

In some embodiments, the dosage of the composition effective in inhibiting growth of pathogenic bacteria and/or promoting growth of beneficial bacteria in a subject is the minimum dosage required (i) to inhibit the growth of the target pathogen(s), (ii) to be lethal to the target pathogen(s), (iii) to increase the growth of the specified production animal in the poultry, livestock, and aquaculture sectors, (iv) to increase the survival of infected animals in the poultry, livestock, and aquaculture sectors, (v) to alleviate the symptoms of infection(s) in the oral, digestive and urinary tracts in humans, or any such application or benefit arising from an improvement in immune response or resistance to the presence of or exposure to pathogens.

In some embodiments, the subject is human or animal.

In some embodiments, the pathogenic bacteria is selected from the group consisting of species of Vibrio, Tenacibaculum, Clostridia, Salmonella, Streptococcus, Aeromonas, Campylobacter, Bacillus, Klebsiella, Listeria, Shigella, Escherichia coli and Piscirickettsia salmonis or pathogenic bacteria containing Type I fimbriae.

In some embodiments, the growth of the pathogenic bacterial is inhibited by at least 20%. In some embodiments, the growth of the pathogenic bacterial is inhibited by at least 30%. In some embodiments, the growth of the pathogenic bacterial is inhibited by at least 40%, at least 50%, at least 75%, or at least 95%, and values therebetween.

In some embodiments, the composition further comprises antibiotics. In some embodiments, the MOS increases the efficacy of the antibiotics. In some embodiments, the MOS increases the efficacy of the antibiotics by at least 30%, by at least 40%, by at least 60%, and values therebetween. In some embodiments, the antibiotics is selected from penicillin such as amoxicillin, gentamycin, clindamycin, kanamycin, tetracycline, erythromycin, ciprofloxacin, vancomycin and ceftazidime. In some embodiments, the antibiotics is penicillin.

Examples of suitable Clostridia species include, but are not limited to perfringens, tetani, sordelii and botulinum. In some embodiments, the Clostridia species are Clostridium perfringens. In some embodiments, the growth of Clostridium perfringens is inhibited by at least 20%. In some embodiments, the growth of Clostridium perfringens is inhibited by at least 30%. In some embodiments, the growth of Clostridium perfringens is inhibited by at least 40%. In some embodiments, the growth of Clostridium perfringens is inhibited by at least 50%.

Examples of suitable Salmonella species include enterica and bongori. In some embodiments, the Salmonella species are Salmonella enteritidis. In some embodiments, the growth of Salmonella enteritidis is inhibited by at least 20% In some embodiments, the growth of Salmonella enteritidis is inhibited by at least 30%. In some embodiments, the growth of Salmonella enteritidis is inhibited by at least 40%. In some embodiments, the growth of Salmonella enteritidis is inhibited by at least 50%.

Examples of suitable Tenacibaculum species include but are not limited to maritimum, soleae, discolor, gallaicum, and dicentrarchi. In some embodiments, the Tenacibaculum species are Tenacibaculum maritimum. In some embodiments, the growth of Tenacibaculum maritimum is inhibited by at least 20%. In some embodiments, the growth of Tenacibaculum maritimum is inhibited by at least 30%. In some embodiments, the growth of Tenacibaculum maritimum is inhibited by at least 40%. In some embodiments, the growth of Tenacibaculum maritimum is inhibited by at least 50%.

Examples of suitable Vibrio species include but are not limited to parahaemolyticus, aguillarum and harveyi. In some embodiments, the Vibrio species are Vibrio parahaemolyticus. In some embodiments, the growth of Vibrio parahaemolyticus is inhibited by at least 20% In some embodiments, the growth of Vibrio parahaemolyticus is inhibited by at least 30%. In some embodiments, the growth of Vibrio parahaemolyticus is inhibited by at least 40%. In some embodiments, the growth of Vibrio parahaemolyticus is inhibited by at least 50%.

In some embodiments, the Vibrio species are Vibrio aguillarum. In some embodiments, the growth of Vibrio aguillarum is inhibited by at least 20% In some embodiments, the growth of Vibrio aguillarum is inhibited by at least 30%. In some embodiments, the growth of Vibrio aguillarum is inhibited by at least 40%. In some embodiments, the growth of Vibrio aguilarum is inhibited by at least 50%.

In some embodiments, the Vibrio species are Vibrio harveyi. In some embodiments, the growth of Vibrio harveyi is inhibited by at least 20% In some embodiments, the growth of Vibrio harveyi is inhibited by at least 30%. In some embodiments, the growth of Vibrio harveyi is inhibited by at least 40%. In some embodiments, the growth of Vibrio harveyi is inhibited by at least 50%.

In some embodiments, the growth of Piscirickettsia salmonis is inhibited by at least 20%. In some embodiments, the growth of Piscirickettsia salmonis is inhibited by at least 30%. In some embodiments, the growth of Piscirickettsia salmonis is inhibited by at least 40%. In some embodiments, the growth of Piscirickettsia salmonis is inhibited by at least 50%. In some embodiments, the growth of Piscirickettsia salmonis is inhibited by at least 70%, at least 80%, at least 90%, or about 100% and values therebetween.

Examples of suitable Streptococcus species include but are not limited to mutans, anginosus, pyogenes, agalactiae and dysgalactieae. In some embodiments, the Streptococcus species are Streptococcus mutans. In some embodiments, the growth of Streptococcus mutans is inhibited by at least 20% In some embodiments, the growth of Streptococcus mutans is inhibited by at least 30%. In some embodiments, the growth of Streptococcus mutans is inhibited by at least 40%. In some embodiments, the growth of Streptococcus mutans is inhibited by at least 50%.

In some embodiments, the growth of Escherichia coli is inhibited by at least 20%. In some embodiments, the growth of Escherichia coli is inhibited by at least 30%. In some embodiments, the growth of Escherichia coli is inhibited by at least 40%. In some embodiments, the growth of Escherichia coli is inhibited by at least 50%.

Examples of suitable Listeria species include but are not limited to monocytogenes, aquatica and seeligeri. In some embodiments, the Listeria species are Listeria monocytogenes. In some embodiments, the growth of Listeria monocytogenes is inhibited by at least 20%. In some embodiments, the growth of Listeria monocytogenes is inhibited by at least 30%. In some embodiments, the growth of Listeria monocytogenes is inhibited by at least 40%. In some embodiments, the growth of Listeria monocytogenes is inhibited by at least 50%.

Examples of suitable Staphylococcus species include but are not limited to aureus, auricularis, borealis, caprae, cohnii, devriesei, gallinarum, hyicus, lentus and sciuri.

Examples of suitable Aeromonas species include but are not limited to hydrophila, caviae, salmonocida, and veronii.

Examples of suitable Campylobacter species include but are not limited to jejuni, coli, upsaliensis, fetus venerealis and lari.

Examples of suitable Bacillus species include but are not limited to cereus, subtills, anthacis and licheniformis.

Examples of suitable Klebsiella species include pneumoniae and others.

Examples of suitable Shigella species include but are not limited to flexneri, sonnei and dysenteriae.

Examples of suitable pathogenic bacteria containing Type I fimbriae include but are not limited to Neisseria and Actinomyces.

A skilled person will understand that the time and extent of a pathogen's inhibition will depend on various factors, such as the dosage of the composition, formulation of the composition, frequency of administration, pathogen's characteristics such as it's doubling time, and others.

In some embodiments, the composition of the present application has an effect on the growth performance of animals when animals are fed with the composition. In some embodiments, growth performance includes at least one of growth rate and Feed Conversion Ratio (FCR). In some embodiments, the composition provides increased growth rate. In some embodiments, the composition provides reduced FCR.

In some embodiments, the composition of the application increases the activity of one or more of IFN-γ, hepcidin, and TLR-9 genes. As such, the composition of the application by upregulating IFN-γ prevents infection and by upregulating hepcidin and TLR-9, helps support the cellular immune response and reduce replication of pathogenic bacteria.

In some embodiments, the composition of the application can be used as a vaccine adjuvant.

The present application also includes a combination of the manno-oligosaccharide (MOS) carbohydrates composition and an antibiotic, wherein the efficacy of the antibiotic is increased by at least 30%.

In some embodiments, the combination provides a synergistic effect of enhanced performance of the antibiotics. In some embodiments, the efficacy of the antibiotic is increased by at least 40%, by at least 50%, by at least 60%, or by at least 70%, and values therebetween.

The content of the MOS and the ratio of the MOS to the antibiotic will vary depending upon the antibiotic and its molecular weight. In some embodiments, the content of the MOS in the combination is from about 1 wt % to about 25 wt %. In some embodiments, the content of the MOS in the combination is about 1 wt %, about 2 wt %, about 3 wt %, about 5 wt %, about 10 wt %, or about 25 wt %, and values therebetween. The MOS in the composition improves the efficacy of the antibiotic.

In some embodiments, the MOS composition comprising manno-oligosaccharide (MOS) carbohydrates wherein at least 70% wt of the MOS carbohydrates are mannose sub-units.

In some embodiments, at least 75% wt of the MOS carbohydrates are mannose sub-units. In some embodiments, at least 80% wt of the MOS carbohydrates are mannose sub-units. In some embodiments, at least 85% wt, or at least 90% wt, or at least 95% wt, of the MOS carbohydrates are mannose sub-units. In some embodiments, at least 85% wt of the MOS carbohydrates are mannose sub-units.

In some embodiments, the MOS is derived from mannan material provided from plant sources selected from palm kernel cake, coconut residue, softwoods such as pine or spruce, residuals from coffee processing, acai seeds and residues, and copra meal. In some embodiments, the mannan material is provided from copra meal.

The antibiotic includes any antibiotic known in the art. Examples of antibiotics include but are not limited to penicillin, gentamycin, clindamycin, ceftazidime and the like. As such, in some embodiments, the antibiotics is selected from penicillin such as amoxicilin, gentamycin, clindamycin, kanamycin, tetracycline, erythromycin, ciprofloxacin, vancomycin and ceftazidime. In some embodiments, the antibiotic is penicillin.

In some embodiments, the degree of polymerization (DP) of the MOS is less than 20. In some embodiments, the DP of the MOS is less than 8, or from 1 to 8. In some embodiments, the DP is from 2 to 10. In some embodiments, the DP is from 2 to 6.

In some embodiments, the MOS having DP of 2 is present in the composition at a content of over 50 wt %. In some embodiments, the MOS having DP of 2 is present in the composition at a content of about 60 wt %, about 65 wt %, about 70 wt %, or about 80 wt %, and values therebetween.

In some embodiments, the combination composition is in a form of an aqueous solution or powder.

In some embodiments, the aqueous solution or powder is provided by hydrolysis and purification of a mannan material and addition of the antibiotics.

In some embodiments, the combination composition is formulated for oral administration.

In some embodiments, the combination composition is formulated in a form of a supplement, food, beverage or feed additive.

In some embodiments, the combination composition is formulated in a form of a capsule, tablet, sachet, or liquid.

In some embodiments, the subject is human or animal.

In some embodiments, the pathogenic bacteria is selected from the group consisting of species of Vibrio, Tenacibaculum, Clostridia, Salmonella, Streptococcus, Aeromonas, Campylobacter, Bacillus, Klebsiella, Listeria, Shigella, Escherichia coli and Piscirickettsia salmonis or pathogenic bacteria containing Type I fimbriae. In some embodiments, the pathogenic bacteria is selected from species of Streptococcus. In some embodiments, the pathogenic bacteria is Streptococcus mutans.

The present application also includes a supplement, food, beverage or feed containing the combination composition of the application.

The present application further includes a capsule, tablet, sachet or liquid containing the combination composition of the application.

In some embodiments, the loading of the composition or the combination composition is less than 1000 mg per 100 gr of the product, such as supplement, food, beverage, feed in a formulation such as a capsule, a tablet or sachet or liquid. In some embodiments, the loading is less than 800 mg per 100 gr or less than 600 mg per 100 gr. The loading can vary depending on factors such as the formulation, the subject to be treated, the age and the sensitivity of the subject and the optimization of the product and the loading of the composition or the combination composition of the application is within the skill of the person skilled in the art.

Supplement, food, beverage, feed or a capsule, a tablet, sachet or liquid containing the composition or the combination composition of the application can be administered at least once a week, from about one time per three weeks, or about one time per week to about once daily for a given treatment. In some embodiments, the supplement, food, beverage, feed or a capsule, a tablet, sachet or liquid containing the composition or the combination composition of the application can be administered 2, 3, 4, 5 or 6 times daily.

### Methods of the Application

The present application also includes a method of producing MOS carbohydrates composition comprising subjecting mannan material to hydrolysis to obtain a crude extract, and purifying the crude extract to obtain a purified extract, wherein at least 70% wt of the MOS carbohydrates are mannose sub-units.

In some embodiments, the mannan material is provided from plant sources that contain-mannan, including but are not limited to palm kernel cake, coconut residue, softwoods such as pine or spruce, residuals from coffee processing, and acai seeds and residues, copra meal and the like. As such, in some embodiments, the MOS is derived from mannan material provided from plant sources selected from palm kernel cake, coconut residue, softwoods such as pine or spruce, residuals from coffee processing, acai seeds and residues, and copra meal. In some embodiments, the mannan material is provided from copra meal.

In some embodiments, the mannan material is hydrolyzed at about 5 to about 30% w/v concentration in a mixture of the mannan material and the enzyme. In some embodiments, the mannan material is hydrolyzed at about 10% w/v, about 15% w/v, about 20% w/v, about 25% w/v or about 30% w/v concentration in a mixture of the mannan material and the enzyme. In some embodiments, the mannan material is hydrolyzed at about 15% w/v concentration in a mixture of the mannan material and the enzyme.

The mannan material is subjected to hydrolysis using hydrolysis methods such as acid hydrolysis, thermal hydrolysis, enzyme hydrolysis, microbial fermentation hydrolysis, and combinations of such methods. Thermal hydrolysis may be conducted at a temperature of about 150° C. to about 220° C. In some embodiments, the hydrolysis is an enzyme hydrolysis.

Hydrolysis of mannan material generates short chain manno-oligosacharids that are further degraded to monosaccharides. Therefore, in the enzyme hydrolysis any enzyme that is capable of hydrolyzing mannan can be used. Examples of such enzyme include but are not limited to β-mannanase, β-mannosidase, β-glucosidase, β-glucanases, β-xylosidase, endo- or exo-xylanases and combinations thereof. It is understood that the concentration of the enzymes is affected by their specific activity and purity, wherein an enzyme with a higher intrinsic/specific activity and/or purity will require a lower dose/concentration, and an enzyme with a lower specific activity and/or purity will require a higher dose/concentration. The selection of the enzyme concentration based upon its specific activity and purity is within the skill of one skilled in the art. Where lower concentration is selected for the hydrolysis, further purification of the MOS may be needed.

In some embodiments, the enzyme is a mixture of β-mannanase and β-mannosidase enzymes. In some embodiments, the enzyme concentration is from about 0.01 to about 0.5% w/v. In some embodiments, the enzyme concentration is about 0.1% w/v.

In some embodiments, the hydrolysis is conducted at temperature of about 40° C. to about 70° C. In some embodiments, the hydrolysis is conducted at temperature of about 50° C. to about 60° C. In some embodiments, the hydrolysis is conducted at temperature of about 60° C. As will be appreciated by those skilled in the art, the reaction time is dependent on the reaction temperature and enzyme concentration/activity, with higher temperatures and higher enzyme concentration/activity favoring more rapid reaction. In some embodiments, the reaction time of the hydrolysis is from about 2 hours to about 12 hours, from about 4 hours to about 10 hours, or from about 6 to about 9 hours.

In some embodiments, the resulting crude extract comprising at least one of monosaccharide selected from the group consisting of mannose, glucose, xylose, arabinose and galactose, oligosaccharides, proteins, polyphenols and lignin-derived compounds, fats/oils, ash, extractives. In some embodiments, the composition of the present application is free from fructose.

In some embodiments, the crude extract is purified by any method known in the art, such as centrifugation, filtration, extraction, absorption, ion exchange, and chromatographic separation, and the like. In some embodiments, the crude extract is purified by filtration. In some embodiments, the purified extract is further purified by cooling the crude extract to a temperature of less than 40° C. and the fat layer is decanted to obtain a fat-free extract.

In some embodiments, the fat-free extract is further purified to remove high and low molecular weight fractions and polyphenols. The purification can be done by any method known in the art. In some embodiments, the fat-free extract is purified through ultrafiltration followed by nanofiltration steps in diafiltration mode to obtain purified extract. Diafiltration mode refers to addition of water to improve recovery. The purification methods can vary and are within the consideration of those skilled in the art.

In some embodiments, the resultant purified extract is concentrated using any method known in the art, e.g., evaporation and reverse osmosis to obtain purified concentrated extract to obtain the MOS composition in a form of an aqueous solution or powder. In some embodiments, the extract is concentrated using multiple-effect evaporator.

In some embodiments, when the MOS composition is in a form of an aqueous solution, the extract may be concentrated to at least 15% wt soluble solids. In some embodiments, the extract may be concentrated to at least 20% wt, at least 30%, at least 40% or at least 50% soluble solids.

In some embodiments, the resultant concentrated purified extract is used as is, or dried into a powder form via any method known in the art, including e.g., refractance window drying, freeze drying, spray drying, fluidized bed drying, and the like.

In some embodiments, the degree of polymerization (DP) of the MOS is less than 20. In some embodiments, the DP of the MOS is less than 8. In some embodiments, the DP is from 2 to 10. In some embodiments, the DP is from 2 to 6.

In some embodiments, the MOS having DP of 2 is present in the composition at a content of over 50 wt %. In some embodiments, the MOS having DP of 2 is present in the composition at a content of about 60 wt %, about 65 wt %, about 70 wt %, or about 80 wt %, and values therebetween. In some embodiments, the method of the application provides MOS carbohydrates with a high content of MOS having DP of 2.

In some embodiments, at least 75% wt of the MOS carbohydrates are mannose sub-units. In some embodiments, at least 80% wt of the MOS carbohydrates are mannose sub-units. In some embodiments, at least 85% wt of the MOS carbohydrates are mannose sub-units.

In some embodiments, the composition of the present application has a water solubility of above 90% at 15 wt % to 25 wt % aqueous solution of the composition at 25° C. In some embodiments, the composition has a water solubility of above 95% at 15 wt % to 25 wt % aqueous solution of the composition at 25° C. In some embodiments, the composition has a water solubility of about 95% to about 100% at 15 wt % to 25 wt % aqueous solution of the composition at 25° C. In some embodiments, the composition has a water solubility at least 95%, at least 96%, at least 97%, at least 98%, or at least 99% at 15 wt % to 25 wt % aqueous solution of the composition at 25° C. In some embodiments, the composition has a water solubility of about 100% at 15 wt % to 25 wt % aqueous solution of the composition at 25° C. Methods to detect and/or quantify water solubility are well known in the art. In some embodiments, the composition of the application wherein the MOS having DP of 2 is present in the composition at a content of over 50 wt % provides improved solubility of the composition.

In some embodiments, the mannose sub-units comprise predominantly β-1,4 linkages. In some embodiments, the β-1,4 linkages of the mannose sub-units are well suited for the utilization by the beneficial bacteria. As such, in some embodiments, the amount of the beneficial bacteria will increase, and may crowd out pathogenic bacteria. The beneficial bacteria are any beneficial bacteria known in the art, such as for example probiotics which include microorganisms such as Lactobacillus, Bifidobacterium, Saccharomyces, Streptococcus, Enterococcus, Escherichia, and Bacillus and the like. In some embodiments, the beneficial bacteria include microorganisms that contain endo, b-1,4 mannanases.

In some embodiments, the composition further comprising at least one monosaccharide selected from the group consisting of glucose, galactose, xylose, arabinose, and combinations thereof. In some embodiments, the composition of the present application is free from fructose.

In some embodiments, the content of glucose is less than 10% wt of the MOS. In some embodiments, the content of glucose is less than 8% wt of the MOS. In some embodiments, the content of glucose is between 3 and 7% wt of the MOS. In some embodiments, the content of glucose is less than 3% wt of the MOS.

In some embodiments, the content of galactose is less than 5% wt of the MOS. In some embodiments, the content of galactose is between 1 and 3% wt of the MOS. In some embodiments, the content of galactose is less than 1% wt of the MOS.

In some embodiments, the composition further comprising glucose and galactose. In some embodiments, the total glucose and galactose content is less than 10% wt of the MOS.

In some embodiments, the total monosaccharide content is less than 15% wt of the MOS. In some embodiments, the total monosaccharide content is less than 13% wt of the MOS. In some embodiments, the total monosaccharide content is less than 10% wt of the MOS.

In some embodiments, the composition further comprises β-glucan, wherein the content of the β-glucan is from about 0.5% wt to about 5% wt of the MOS. In some embodiments, the composition further comprises β-glucan wherein the content of the β-glucan is about 1%, about 2%, about 3%, or about 4% of the MOS, and values therebetween. The following examples illustrate the invention. These examples should not be interpreted as limiting the invention, but are illustrative of the invention, its beneficial properties and certain embodiments.

### EXAMPLES

### General Methods and Materials

Carbohydrates were analyzed using high-performance liquid chromatography (HPLC) with refractive index detection. NREL Laboratory Analytical Procedures (LAPs) were used to determine structural carbohydrates, lignin, ash, extractives (NREL TPs 510-42622, 510-42625, 510-42619, 510-42618, 510-42623).

### Example 1: Production of High Purity β-MOS

Copra meal was hydrolyzed at 15% (w/v) concentration in a mixture of β-mannanase and β-mannosidase enzymes at 0.1% (w/v) concentration. The slurry was left for 8 h at 60° C. to obtain a hydrosylate. The resulting hydrolysate contained unconverted solids, mannose, short-chain and medium chain manno-oligosaccharides, other carbohydrates (as monomers and oligosaccharides), oil/fat, soluble polyphenols, ash and other extractives.

The liquid was recovered from the hydrolysate via a series of solid filtration steps, resulting in a clear liquid. Residual oil was removed by cooling the extract to 20° C. and decanting the fat layer.

The fat-free extract was processed through a series of ultrafiltration (4 kDa molecular weight cut off (MWCO)) and nanofiltration (450 kDa MWCO) steps in diafiltration mode to remove high and low molecular weight fractions and polyphenols, and the nanofiltration retentate was concentrated to 18 wt % soluble solids before spray drying. Alternatively, the purified extract may be concentrated to at least 50% by evaporation, e.g., in a multiple-effect evaporator.

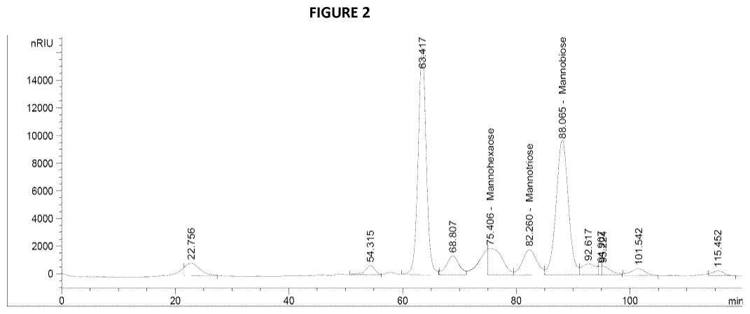

The purified extract was analyzed using HPLC with refractive index detection to identify the DP of the carbohydrates. Over 60% of the mannooligosaccharides showed DP from 2-6, as can be seen on FIG. 2. Mannose elutes at around 101 minutes. The large peak at 63 minutes and smaller peak at 68 minutes are other oligosaccharides with a higher degree of polymerization-likely manno-oligosacchides in the range of DP7 to DP10.

The solubility of the dried powder was at least 20 g in 100 g of water at 25° C.

The concentrated liquid or dried powder (<5% moisture) was used in subsequent trials to assess the efficacy of the product.

### Example 2: In Vitro Study to Assess Inhibition of Salmonella enteritidis (SE)

A 100 μL aliquot of SE in tris buffered saline (TSB) (1.0E+06 CFU (colony forming unit)/mL) and a 100 μL aliquot of cell free supernatant (CFS) were dispensed into individual microtiter plate wells.

Two controls were evaluated:
1) 100 μL of SE (1.0E+06 CFU/mL) and 100 μL of sterile media (TSB; tryptic soy broth) (positive control).
2) 100 μL of sterile media and 100 μL of sterile 0.85% saline (media control)

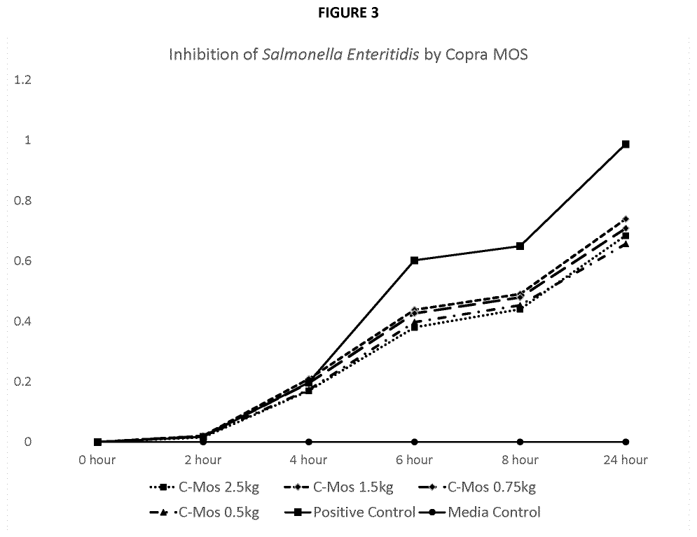

High purity, β-MOS was added as a dry powder to the media in the treatment plates, at doses ranging from an equivalent dose in feed of 0.05 wt % to 0.25 wt %. The microtiter plates were read at 630 nm every 2 h over 24 h to obtain optical density (OD) measurements, while maintaining the temperature at 37° C.±2° C. Results in FIG. 3 represent the average OD measurements of three microtiter wells.

There was a consistent reduction in growth of Salmonella enteritidis (i.e., inhibition) when the high purity β-MOS from copra meal of the present application was added to the medium. The degree of inhibition was 29% at 6 h, 30% at 8 h, and 32% after 24 h. There was no clear dose dependence.

### Example 3: In Vitro Study to Assess Inhibition of Clostridium perfringens (CP)

A 100 μL aliquot of CP in thioglycolate with beef extract (1.0E+08 CFU (colony forming unit)/mL) and a 100 μL aliquot of cell free supernatant (CFS) were dispensed into individual microtiter plate wells.

Two controls were evaluated:
1) 100 μL of CP (1.0E+08 CFU/mL) and 100 μL of sterile media (positive control)
2) 100 μL of sterile media and 100 μL of sterile 0.85% saline (media control)

High purity, β-copra-MOS of the present application and yeast-“MOS” were added as a dry powder to the media in the treatment plates, at doses ranging from an equivalent dose in feed of 0.05 wt % to 0.25 wt %. The yeast-“MOS” remained as a suspension. This is consistent with the specification for yeast MOS products (The Blocking Effect on Undesirable Bacteria, Product Monograph from Lallemand Animal Nutrition, lallemandanimalnutrition.com). The low solubility of yeast-“MOS” made it difficult to accurately discern cells from particles of yeast-“MOS”. The microtiter plates were read at 630 nm at Oh and 18 h to obtain optical density (OD) measurements, while maintaining the temperature at 37° C.±2° C. under anaerobic conditions. Results represent the average OD measurements of three microtiter wells.

There was a 42% reduction after 18 h in growth of C.P. (i.e., inhibition) when the high purity β-MOS from copra meal of the present application was added to the medium. There was no clear dose dependence. Addition of yeast-“MOS” led to particulates/precipitate in the medium; although the OD was less at 18 h compared to the positive control, it is not clear if this is due to a difference in growth or due to particulates from the additive.

### Example 4: In Vitro Study to Assess Inhibition of Vibrio parahaemolyticus and Tenacibaculum maritimum Using Yeast-“MOS” (YMOS) and High Purity Copra-MOS (CMOS)

Powdered CMOS and YMOS were added to distilled water to stock solutions of 400 mg/mL and solubility was determined, including post-centrifugation at 5,000 g for 5 minutes. YMOS was found to be insoluble and so an additional test group, YMOS partially dissolved in 0.8% 1M NaOH solution (YMOS NaOH) was added to the study. Test concentrations of each solution were prepared by serial dilution, with concentrations ranging from 0.21 to 50 mg/mL.

Trials with Tenacibaculum maritimum were conducted over 7-10 days at 15° C. in marine agar/marine broth (MA/MB) media. Trials with Vibrio parahaemolyticus were conducted over 24 h at 37° C. in tryptone soy agar/tryptone soy broth (TSA2/TSB2) media.

Broth cultures were diluted 1:50 in respective broth media, mixed, and 100 μL pipetted into 96-well plates. Compound solutions were added in quadruplicate at 2× final concentration to achieve the effective concentrations desired. Each well contained a 1:1 mixture of compound and pathogen for a 200 μL total volume of 1:100 diluted pathogen and the effective compound concentration. Negative controls (excluding product) and blank controls (excluding pathogen) were prepared. The optical density (OD) was measured at 600 nm, and the change in OD was calculated. The initial bacterial concentration was quantified by plating diluted broth on agar.

The minimum inhibitory concentration (MIC) was calculated using the OD data. The MIC represents the lowest concentration of the product that leads to a statistically significant reduction in OD relative to the negative (product-free) control and inhibits pathogen growth by at least 20%.

The minimum lethal concentration (MLC) was determined by quantifying the growth on agar plates. The MLC represents the lowest concentration of product that led to no bacterial growth on the agar plate.

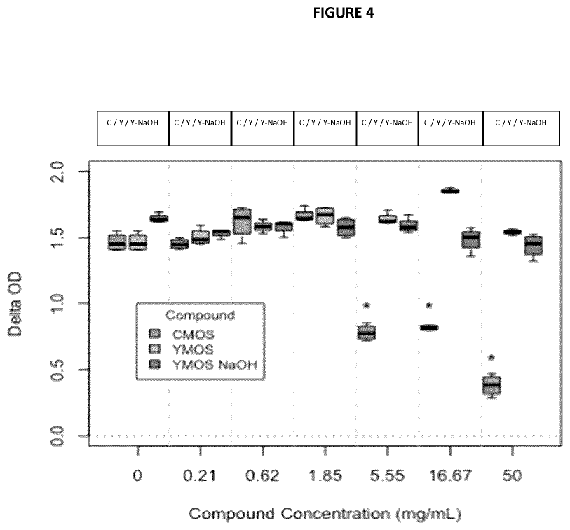

FIG. 4 shows the change in optical density (OD) for each of the compound solutions, at different concentrations in Vibrio parahaemolyticus. Only the CMOS product, at 5.55 mg/mL, 16.67 mg/mL, and 50 mg/mL led to a statistically significant reduction in the growth of Vibrio parahaemolyticus.

TABLE 1
shows the MIC and MLC data for the products.

| | MIC (mg/mL) | | | MLC (mg/mL) | | |
|---|---|---|---|---|---|---|
| | | | YMOS- | | | YMOS- |
| Pathogen | CMOS | YMOS | NaOH | CMOS | YMOS | NaOH |
| Vibrio | 5.55 | ND | ND | 50 | ND | ND |
| parahaemolyticus | | | | | | |
| Tenacibaculum | 16.67 | 50 | ND | 50 | 50 | ND |
| maritimum | | | | | | |

ND = could not be determined, i.e., the MIC/MLC is above 50 mg/mL
CMOS = high purity, β-MOS produced from Copra Meal
YMOS = low purity, α-MOS (mannan) product produced from Yeast Cell Walls. Product also includes β-glucan and protein, among other impurities.

Evaluation of the change in optical density (OD) for each of the products in Tenacibaculum maritimum indicated that Y-MOS NaOH enhanced the growth of Tenacibaculum maritimum, contrary to the desired/intended effect. This result may be due to impurities in the α-mannan/α-MOS compound “solution”. The lower MIC value for the high purity, β-MOS (CMOS) shows that it was more effective against Tenacibaculum maritimum than the low purity α-mannan/α-MOS product from yeast (YMOS). The MLC values were comparable. Solubilizing the α-mannan/α-MOS product using NaOH did not improve the MIC and MLC values, and indeed, made the MLC value worse relative to the MLC values for CMOS and YMOS. Thus, while solubility may be a contributing factor to the efficacy of CMOS, enhancing the solubility of YMOS was unable to improve its performance.

The lower MIC and MLC values for the high purity, β-MOS (CMOS) shows that it was more effective against Vibrio parahaemolyticus than the low purity α-mannan/α-MOS product from yeast (YMOS). Indeed, even a YMOS concentration of 50 mg/ml (nearly 10× higher than the MIC for CMOS) was unable to inhibit the growth of this pathogen. By comparison, there is only a 2.5 to 3-fold difference in purity and MOS/mannan content between CMOS and YMOS. Furthermore, the efficacy of the CMOS product is observed in spite of the absence of β-glucan, which is also reported to possess bioactive properties and potential antimicrobial activity [Amer E. M., Enhancement of β-Glucan Biological Activity Using a Modified Acid-Base Extraction Method from Saccharomyces cerevisiae, Molecules 2021, 26 (8), 2113].

### Example 5: In Vitro Study to Assess Inhibition of Vibrio anguillarum Using Yeast-“MOS” (YMOS) and High Purity, Copra-MOS (CMOS)

This example was conducted in a similar manner to Example 4, using CMOS and YMOS incubated with Vibrio anguillarum for 24 h at 20° C., using TSA2/TSB2 media.

CMOS and YMOS at 50 mg/mL were both effective at inhibiting the growth of Vibrio anguillarum; CMOS inhibited growth by 49%, and YMOS inhibited growth by 32%.

### Example 6: In Vitro Study to Assess Inhibition of Piscirickettsia salmonis

Atlantic Salmon Kidney (ASK) cells were grown in sterile L15 media (15% fetal bovine serum, 20 mM N-2-hydroxyethylpiperazine-N-2-ethane sulfonic acid (HEPES)) and grown to confluence. 1M sodium hydroxide and 1M acetic acid were added to the media to bring the pH within a range from 7.0 to 7.6. Designated amounts of each of two products-Copra MOS (CMOS) and Yeast MOS (YMOS) were added. Product-free controls were also prepared. ASK cells were monitored daily to assess potential cell toxicity, measured as the mean cytopathic effect (CPE).

YMOS was toxic to kidney (ASK) cells at concentrations of 5.55 mg/mL and above, leading to cell shredding and dissociation. CMOS led to mild toxicity at a dose of 50 mg/mL, and had no adverse effects at 1.11, 5.55, and 16.67 mg/mL. This illustrates the clear difference in safety between a high purity soluble β-MOS formulation and a low purity insoluble α-MOS formulation that includes β-glucan, protein, and other components.

In a counterpart study, ASK cells were grown as described above. P. salmonis was diluted in L15 media and added to the test wells along with ASK cells. Wells contained CMOS, YMOS, or MOS-free controls. The ASK cells were monitored to assess the cytopathic effect induced by P. salmonis, in the presence of YMOS or CMOS, or in MOS-free controls. Due to the toxicity of YMOS to ASK cells noted above, YMOS was only tested at 1.85 mg/mL, whereas CMOS was tested at 0.67, 1.85, 5.55, 16.67, and 50 mg/mL.

YMOS at 1.85 mg/mL had no discemable effect on P. salmonis infection in ASK cells after 19 days.

CMOS at 16.67 mg/mL reduced the cytopathogenic effect of P. salmonis by 51% after 19 days. CMOS from 0.67-5.55 mg/ml had no discernable effect on P. salmonis progression (Table 2). Results with CMOS at 50 mg/mL were obscured by cytotoxicity of the product on ASK cells at this concentration.

TABLE 2
Cytopathogenic Effect (CPE) and Inhibition
of P. Salmonis by High Purity Copra β-MOS

| | Observation Timepoint | | | | | |
|---|---|---|---|---|---|---|
| CMOS | (Mean % CPE) | | | | | |
| Concentration | 7 Days | | 13 Days | | 19 Days | |
| (mg/mL) | Control | Experimental | Control | Experimental | Control | Experimental |
| 50.00 | 10.00 | 5.00 | 33.75 | NA | 81.25 | NA |
| 16.67 | 11.25 | 7.50 | 36.25 * | 15.00 * | 93.75 * | 42.50 * |
| 5.55 | 13.25 | 11.25 | 37.50 | 32.50 | 92.50 | 90.00 |
| 1.85 | 15.00 | 13.75 | 47.25 | 43.75 | 95.00 | 97.50 |
| 0.67 | 12.50 | 11.25 | 45.00 | 45.00 | 95.00 | 95.00 |

NA: Not Applicable, CPE obscured by test compound cytotoxicity by Day 11
* Statistically significant inhibition (p < 0.05, inhibition >20.00% between experimental and control)

### Example 7: In Vivo Study to Assess Survival of Shrimp Following Exposure to Vibrio parahaemolyticus

A concentrated liquid preparation of copra-derived β-MOS (CMOS) was used for a Vibrio challenge study in white-legged shrimp. The liquid preparation was added to feed pellets via top-coating. The target doses were 0.25 wt % and 0.50 wt % in the feed.

Twelve shrimp were allocated to each of five tanks for four different groups: (i) a negative control group that was not exposed to Vibrio; (ii) a positive control group exposed to Vibrio but not treated with CMOS, and (iii) two groups treated with CMOS, at a nominal dose of “0.25 wt %” and “0.50 wt %”.

Except for the negative control group, shrimp in the other three groups were exposed to Vibrio paramaemolyticus daily over 8 days. The dose was low for the first three days, then doubled on days 4 and 5, and increased again by a further factor of 2.5 on day 6. Survival of shrimp in each group was monitored daily.

Table 3 illustrates the average survival data for shrimp that received CMOS relative to the positive control exposed to Vibrio but without MOS, and the negative control group that was not exposed to Vibrio.

TABLE 3
Impact of Copra-derived β-MOS (CMOS) on
survival of white-legged shrimp following a
challenge with Vibrio paramaemolyticus

| | Control | Control | |
|---|---|---|---|
| | (−) | (+) | |
| | Neg. | Pos. | |
| | Control | Control | |
| Time | (No | (no | CMOS |
| [days] | Vibrio) | MOS) | (aggregate) |
| 0 | 100 | 100.0 | 100.0 |
| 1 | 100 | 100.0 | 100.0 |
| 2 | 100 | 100.0 | 100.0 |
| 3 | 100 | 100.0 | 100.0 |
| 4 | 100 | 100.0 | 100.0 |
| 5 | 100 | 91.7 | 100.0 |
| 6 | 100 | 58.3 | 100.0 |
| 7 | 100 | 50.0 | 100.0 |
| 8 | 100 | 41.7 | 91.7 |

CMOS = high purity, β-MOS produced from Copra Meal

The survival data in Table 3 illustrate the vast improvement in survival of shrimp consuming CMOS following exposure to Vibrio parahaemolyticus, a significant pathogen in the aquaculture industry.

By comparison, Rungrassamee et al. (Mannooligosaccharides from copra meal improves survival of the Pacific white shrimp (Litopenaeus vannamei)) observed a modest increase in survival from about 40% to a range of 50-60%, depending upon the dose, when performing a Vibrio challenge study using low purity MOS. Similarly, Cuong et al. (Bioconversion Of Copra Meal Into Prebiotic Mannooligosaccharides Using Endo-β-1,4-Mannanase Producing By Aspergillus Niger Bk 01, Science and Technology Journal, vol 48 (3), p 43-49 2010) observed an increase in survival from 70% to 97% using a weaker Vibrio challenge study, and required a much higher dose of their crude MOS preparation—1 wt %—versus a dose as low as 0.25 wt % for the study reported in Table 3 above.

### Example 8: In Vivo Study to Assess Growth of Shrimp

A concentrated liquid preparation of copra-derived β-MOS (CMOS) and a powdered preparation of impure yeast-derived β-MOS (YMOS) were used for a growth study in white-legged shrimp. The preparations were added to shrimp feed pellets via top-coating. The target doses for CMOS were 0.25 wt % and 0.50 wt % in the feed, and 0.25 wt % for YMOS.

Five tanks containing 15 shrimp each were set up for each group. Shrimp were fed one of the MOS preparations outlined above, or a feed without MOS. Weights and growth rates were measured over 8 weeks. The feed conversion ratio was measured at 4 weeks. The growth rates were calculated based on measurements from 0-4 weeks, 0-6 weeks, and from 2-8 weeks. The latter calculation excludes the first 2 weeks of growth data, to ensure that the shrimp are in the linear growth phase.

All tanks were on the same recirculating aquaculture system (RAS) that control for nitrification, carbon dioxide and oxygen content, and for solids removal. A heater was used to maintain temperature at temperatures consistent with commercial operations.

Table 4 summarizes the growth rate and feed conversion ratio (FCR) data for the shrimp receiving the different types of MOS.

TABLE 4
Effect of Copra-derived β-MOS (CMOS) and yeast derived
α-mannan/α-MOS (YMOS) on the growth of white-legged shrimp.

| | Weeks 0-4 | Weeks 0-6 | Weeks 2-8 | |
|---|---|---|---|---|
| | average | average | average | |
| | growth | growth | growth | Week 4 |
| Trial Group | rate, g/week | rate, g/week | rate, g/week | FCR |
| 0.25% CMOS | 0.81 | 0.91 | 1.13 | 1.57 |
| 0.50% CMOS | 0.83 | 1.13 | 1.56 | 1.54 |
| 0.25% YMOS | 0.6 | 0.83 | 0.97 | 1.82 |

The trial data in Table 4 indicate consistent, superior growth rates for shrimp consuming CMOS relative to shrimp consuming YMOS, and the superior growth rates of shrimp consuming 0.50% CMOS relative to shrimp consuming 0.25% CMOS. The difference in growth rate for CMOS relative to YMOS is apparent throughout the trial, whereas the difference between the 0.50 wt % CMOS and 0.25 wt % CMOS groups only becomes apparent after ˜6 weeks, and the difference increases when data at weeks 7 and 8 are considered (Weeks 2-8 average growth rates).

The Feed Conversion Ratio (FCR) data for Week 4 also point to superior performance for shrimp consuming CMOS versus shrimp consuming YMOS (a lower FCR is preferred-less feed consumed per unit mass gain of the animal).

### Example 9: Lactobacillus Growth Study

Growth of L. rhamnosus GG (LGG) was evaluated using a range of concentrations, comparing high purity copra-MOS (CMOS), yeast MOS (YMOS), FOS, inulin, XOS, and glucose (positive control). A total of 100 μl of broth is added to each well—90 μl of 100 mg/ml MRS broth with or without 100 mg/ml prebiotic and 10 μl of bacteria pre-cultured to approximately OD=1. The cell density was measured at OD₆₀₀, taking measurements at regular intervals over 24 hours.

As expected, the greatest growth of LGG occurred on glucose (OD=1.6 at 24 h). Growth on FOS and CMOS was comparable (OD˜1.0 for each), and superior to growth on inulin and XOS (OD˜0.6 and 0.7, respectively). Growth on YMOS was indistinguishable from controls grown in MRS media without prebiotics (OD˜0.3).

### Example 10: In Vitro Study to Assess Immune Stimulation of MOS and Inhibition of Piscirickettsia salmonis in Atlantic Salmon Head Kidney (SHK) Cells

SHK-1 cells sourced from ECACC (ECACC 97111106) were grown in sterile supplemented L15 media (5% fetal bovine serum, 20 mM HEPES, 40 μM beta-mercaptoethanol) and were split into 12-well plates to incubate at 20° C. until confluent. 1 M sodium hydroxide and 1M acetic acid were added to bring the pH within a range from 7.0-7.6. Designated amounts of two products—Copra MOS (CMOS) of the present application and Yeast MOS (YMOS) were added. Product-free controls were also prepared. Each treatment and control had a minimum of eight replicates. The initial titer of P. salmonis was validated via TCID₅₀ using a modified Spearman-Karber method known in the art.

The plates were incubated at 15° C. throughout the duration of the assay. Observations were made daily for the first two days and at least every 72 h thereafter for cytopathic effect (CPE) and monolayer dissociation rounded to the nearest 5% until mean CPE and/or dissociation in negative controls reached 35-55% or until the assay lasted for a minimum of 20 days. Minimum Inhibitory Concentration (MIC) was determined by statistically significant (p<0.05) CPE reduction of >20% compared to negative control.

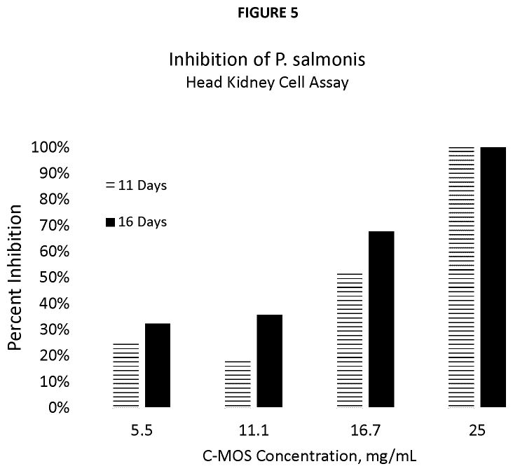

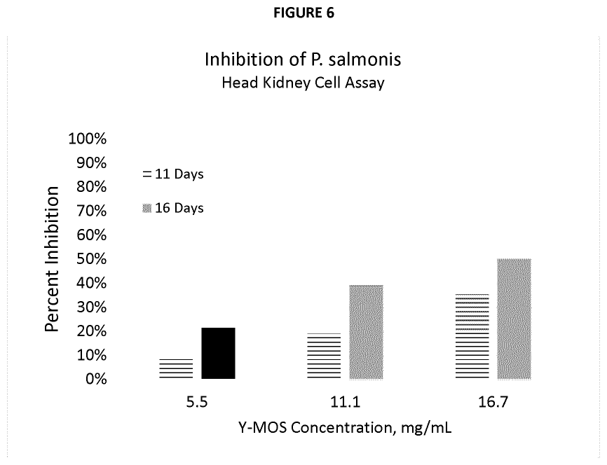

FIGS. 5 and 6 show the inhibition of P. salmonis by copra MOS and yeast MOS. The 25 mg/ml dose of yeast MOS caused cytotoxicity, and full study data could not be collected for this concentration. High concentrations of YMOS (25 mg/ml) resulted in chronic cell stress and eventual dissociation of the cell layer.

Notably, 100% inhibition of P. salmonis was attained after 11 days at 25 mg/mL, and sustained until the end of the study. Greater than 20% inhibition—the criterion for minimum inhibitory concentration—was observed after 11 days at a copra MOS dose of 5.5 mg/mL.

### Example 11: Analysis of Biomarkers

Cells were harvested from the well plates during the studies of minimum inhibitory concentration, then stored at −80° C. prior to RNA extraction. Cells incubated in 16 and 25 mg/ml of CMOS and YMOS were collected at the beginning and end of the MIC assay, along with positive and negative controls. RNA was isolated from selected cell samples and purified in accordance with the manufacturer's instructions using the Qiagen® RNeasy Kit with on-column DNase digestion and the Zymo® Clean & Concentrator Kit. Pure RNA was achieved for all extracted samples. cDNA was synthesized, then used to conduct a set of SYBR-based qPCR assays to evaluate gene expression of genes of interest (campb, cd209, IFN-γ, IL-1β, hepcidin, and TLR-9) and two reference genes (ef1-a and eif-3d) in each sample. A total of 216 sample and gene combinations were analyzed in triplicate.

CMOS altered expression of IFN-γ, hepcidin, and TLR-9 within 3 h of cell exposure to the product. Meanwhile, YMOS altered expression of campb, cd209, IFN-γ, IL-1β, hepcidin, and TLR-9 in cells by the end of the cell assay, but not in a manner that was different from the pathogen control. In contrast, CMOS led to a difference in expression of IFN-γ, hepcidin, and TLR-9 between the cell controls and pathogen controls at study termination.

There was an ongoing immune response triggered by YMOS, coinciding with the observed cytotoxicity and YMOS-induced cell stress. YMOS stimulated a pro-inflammatory immune response via campb, IL-1β, and TLR-9, and YMOS attached to the mannose-binding site of cd209 in SHK cells, which would reduce bacterial replication.

The near immediate and enhanced upregulation of IFN-γ by CMOS points to a primary pathway to prevent infection. Similarly, the upregulation of hepcidin and TLR-9 help support the cellular immune response and reduce replication of P. salmonis within the cells. Long-term immunostimulation of campb was also observed.

### Example 12: Data from Combination of MOS with Antibiotics

A pathogen inhibition study was conducted to compare inhibition of Streptococcus mutans by penicillin vs. a combination of penicillin and the MOS preparation of Example 1. S. mutans was grown in BHI media. Using penicillin alone reduced S. mutans growth by 10%, whereas combining penicillin with 5 wt % MOS inhibited S. mutans growth by 75%. This demonstrates the ability of the MOS composition to enhance the performance of antibiotics.

### Example 13: Effect of MOS DP on Pathogen Inhibition

Pathogen inhibition studies were conducted using Salmonella enteritidis incubated in (i) media, (ii) mannose (iii) manno-oligosaccharides with different degrees of polymerization, including the low DP MOS composition of Example 1, and a MOS mixture prepared with a higher average degree of polymerization. OD₆₀₀ data were obtained for each substrate, and the inhibition (or enhanced growth) relative to the growth control was measured. The relative growth is indicated in Table 5 below. A value less than 1 indicates inhibition; a value greater than 1 indicates growth is promoted by the substrate.

TABLE 5

| | |
|---|---|
| mannose | 0.97 |
| low DP MOS | 0.63 |
| high DP MOS | 1.58 |

The data in Table 5 illustrate the impact of the degree of polymerization of the MOS product. In this case, a MOS mixture with a higher average DP (80% DP4+) promoted the growth of S. enteritidis, whereas a MOS mixture with a lower average DP (64% DP2) inhibited S. enteritidis growth by 37%.

### Example 14: Solubility Measurements

The solubility of the MOS product was evaluated by adding MOS to a specified quantity of water, to make an “X” wt % solution. Solubility was evaluated at ambient temperature (approximately 25° C.), and at 40° C. Two separate compositions of MOS were tested. Composition 1 represents the composition described in Example 1; Composition 2 has a lower average DP, due to the presence of 64% mannobiose (DP2) in the blend.

Composition 1 was completely (100%) soluble at ambient temperature at a concentration up to 15 wt % MOS. Composition 1 was completely (100%) soluble at 40° C. at concentrations up to 17.5 wt % MOS.

Composition 2 was completely (100%) soluble at ambient temperature and at 40° C. at a concentration up to 25 wt %.

1-35. (canceled)

36. A combination of manno-oligosaccharide (MOS) carbohydrates composition and an antibiotic, wherein the efficacy of the antibiotic is increased by at least 30%.

37. (canceled)

38. The combination of claim 36, wherein at least 85% wt of the MOS carbohydrates are mannose sub-units.

39. The combination of claim 36, wherein the MOS is derived from mannan material provided from plant sources selected from palm kernel cake, coconut residue, softwoods such as pine or spruce, residuals from coffee processing, acai seeds and residues, and copra meal.

40. (canceled)

41. The combination of claim 36, wherein the antibiotics is selected from penicillin, amoxicilin, gentamycin, clindamycin, kanamycin, tetracycline, erythromycin, ciprofloxacin, vancomycin and ceftazidime.

42-43. (canceled)

44. The combination of claim 36, wherein the pathogenic bacteria is selected from the group consisting from species of Vibrio, Tenacibaculum, Clostridia, Salmonella, Streptococcus, Aeromonas, Campylobacter, Bacillus, Klebsiella, Listeria, Shigella, Escherichia coli and Piscirickettsia salmonis or pathogenic bacteria containing Type I fimbriae.

45-74. (canceled)

75. A method of inhibiting growth of pathogenic bacteria and/or promoting growth of beneficial bacteria in a subject, comprising administering to the subject a composition comprising manno-oligosaccharide (MOS) carbohydrates wherein at least 70% of the MOS carbohydrates are mannose sub-units.

76. The method according to claim 75, wherein the MOS is derived from mannan material provided from plant sources selected from palm kernel cake, coconut residue, softwoods such as pine or spruce, residuals from coffee processing, acai seeds and residues, and copra meal.

77. The method according to claim 75, wherein the DP is from 2 to 10.

78. The method according to claim 77, wherein the MOS having DP of 2 is present in the composition at a content of over 50 wt %.

79. The method according to claim 75, wherein at least 85% wt of the MOS carbohydrates are mannose sub-units.

80. The method according to claim 75, wherein the composition has a water solubility of above 90% at 15 wt % to 25 wt % aqueous solution of the composition at 25° C.

81. The method according to claim 80, wherein the composition has a water solubility of about 100% at 15 wt % to 25 wt % aqueous solution of the composition at 25° C.

82. The method according to claim 75, wherein the mannose sub-units comprise predominantly b-1,4 linkages.

83. The method according to claim 75, wherein the composition has a total glucose and galactose content of less than 10% wt of the MOS.

84. The method according to claim 75, wherein the composition is free from fructose.

85. The method according to claim 75, wherein the composition further comprises b-glucan, wherein the content of the b-glucan is from about 0.5% wt to about 5% wt of the MOS.

86. The method according to claim 75, wherein the composition further comprises an agent selected from the group consisting of probiotics, prebiotics, polyphenols, phages, clays and minerals such as bentonite and montmorillonite, antibiotics and short chain fatty acids.

87. The method according to claim 75, wherein the pathogenic bacteria is selected from the group consisting of species of Vibrio, Tenacibaculum, Clostridia, Salmonella, Streptococcus, Staphylococcus, Aeromonas, Campylobacter, Bacillus, Klebsiella, Listeria, Shigella, Escherichia coli and Piscirickettsia salmonis, or pathogenic bacteria containing Type I fimbriae.

88. The method according to claim 87, wherein the growth of the pathogenic bacteria is inhibited by at least 20%.

89. The method according to claim 75, wherein the composition further comprises antibiotics.

90. The method according to claim 89, wherein the MOS increases the efficacy of the antibiotics.

91. The method according to claim 89, wherein the antibiotic is selected from penicillin, amoxicilin, gentamycin, clindamycin, kanamycin, tetracycline, erythromycin, ciprofloxacin, vancomycin and ceftazidime.

92. The method according to claim 75, wherein the growth of Clostridium perfringens is inhibited by at least 20%, wherein the growth of Salmonella enteritidis is inhibited by at least 20%, the growth of Tenacibaculum maritimum is inhibited by at least 20%, the growth of Vibrio parahaemolyticus is inhibited by at least 20%, the growth of Vibrio aguillarum is inhibited by at least 20%, the growth of Vibrio harveyi is inhibited by at least 20%, the growth of Piscirickettsia salmonis is inhibited by at least 20%, the growth of Streptococcus mutans is inhibited by at least 20%, the growth of Escherichia coli is inhibited by at least 20% or the growth of Listeria monocytogenes is inhibited by at least 20%.

93. The method according to claim 75, wherein the composition is administered to the subject as a supplement, food, beverage or feedstock.

94. The method according to claim 75, wherein the composition is administered to the subject as a capsule, tablet, sachet or liquid.

95. The method according to claim 75, wherein the composition is administered to the subject as a vaccine adjuvant.
